(19) United States
(12) Patent Application Publication
(10) Pub. No.: US 2012/0238738 A1
(43) Pub. Date: Sep. 20, 2012

(54) Oligonucleotide Adapters: Compositions and Methods of Use

(75) Inventor: Cynthia Hendrickson, Wenham, MA (US)

(73) Assignee: NEW ENGLAND BIOLABS, INC., Ipswich, MA (US)

(21) Appl. No.: 13/513,726

(22) PCT Filed: Jun. 13, 2011
(86) PCT No.: PCT/US11/40173

§ 371 Date: Jun. 4, 2012

Related U.S. Application Data
(60) Provisional application No. 61/365,687, filed on Jul. 19, 2010.
(60) Provisional application No. 61/381,767, filed on Sep. 10, 2010.

(51) Int. Cl. C07H 21/04; C12P 19/34

(52) U.S. Cl. 536/23.1; 435/91.52; 435/91.2

(57) ABSTRACT

Compositions are provided that include a synthetic oligonucleotide characterized by a double-stranded region, a single-stranded region, a forward primer site, a reverse primer site and one or more cleavage sites therebetween. Methods of use for these compositions include adaptors for the amplification of DNA fragments.

### BACKGROUND OF THE INVENTION

Genome mapping commonly relies on amplification of large numbers of nucleic acid sequences preferably in an efficient and unbiased manner using polymerase chain reaction (PCR) amplification. PCR preferably relies on forward and reverse primers, which anneal to DNA sequences that flank a target sequence. Y-shaped adapters and double-stranded DNA universal adapters with internal mismatches have been developed to add known primer sites to DNA of unknown sequence. These Y-adapters share the property of having two separate strands of DNA to form double-stranded and single-stranded regions (U.S. Pat. No. 7,741,463). The separate strands of the double-stranded adapters are ligated to each end of a target sequence and a primer pair is added to the ligated DNA. One primer anneals to a sequence in an adapter at one end of the target DNA and the other primer in the pair anneals to a sequence on the complementary strand of the adapter at the other end of the target DNA. The entire process requires multiple enzymes and multiple distinct reactions, which require purification at various stages.

### SUMMARY OF THE INVENTION

In an embodiment of the invention, a composition is provided that includes a synthetic oligonucleotide, which is characterized by a double-stranded region, a single-stranded region, a forward primer site, a reverse primer site and one or more cleavage sites therebetween and is optionally resistant to exonuclease-degradation. The oligonucleotide optionally remains non-denatured during an enzyme-denaturing temperature.

The synthetic oligonucleotide may be further characterized by a nucleotide sequence that permits the oligonucleotide to fold into a structure having at least one single-stranded loop and one double-stranded region where the double-stranded region has a 3′ end and a 5′ end. In one embodiment, the 3′ end and the 5′ end form a blunt end or a staggered end where the 5′ end may be phosphorylated or adenylated. The one or more cleavage sites in the oligonucleotide may comprise a modified nucleotide or a sequence containing a modified or unmodified nucleotide that is specifically recognized by a cleavage agent, a chemical group, a chemical linker or a spacer. The forward primer site may be positioned between the 3′ end and a proximate cleavage site and the reverse primer site may be positioned between the 5′ end and the same or different proximate cleavage site.

In an embodiment of the invention, the oligonucleotide includes a barcode sequence which is adjacent to a primer site which is complementary to a primer for replicating the barcode. The barcode may be 2-15 nucleotides in length and may be positioned on the oligonucleotide adjacent to a polynucleotide of unknown or known sequence ligated to the oligonucleotide.

In an embodiment of the invention, the oligonucleotide is capable of use as an adapter for amplification and/or sequencing reactions.

In an embodiment of the invention, a method is provided for preparing polynucleotides for ligation that includes blunt-ending a polynucleotide using an enzyme mixture comprising T4 DNA polymerase and a thermostable polymerase, such as for example, an archaeal polymerase, having 3′-5′ exonuclease activity such that the blunt-ended polynucleotide is capable of being ligated to an oligonucleotide of the type described herein. Oligonucleotides may be ligated using a ligase to each end of the blunt-ended polynucleotide. The ligated oligonucleotide can then be cleaved with a cleaving agent.

In an embodiment of the invention, a method is provided for preparing an amplification-ready polynucleotide in a reaction vessel that includes ligating by means of a ligase, a composition described herein to each end of the polynucleotide; treating the preparation of ligation products with a cleaving agent, wherein the cleaving agent cleaves the oligonucleotide at or near the one or more modified components in the oligonucleotide; and amplifying the polynucleotide. The polynucleotidereferred to herein may be DNA or RNA or a DNA/RNA hybrid.

A nuclease may additionally be added to the reaction vessel to degrade unligated polynucleotides and/or unligated oligonucleotides. The reaction vessel is heated to an enzyme-denaturing temperature suitable for denaturing the degrading enzyme and/or ligase. The methods described herein may be performed in a single reaction vessel.

### BRIEF DESCRIPTION OF THE FIGURES

FIGS. 1A-B show different types of universal loop adapters (8 and 9 in FIG. 1A and 10 and 11 in FIG. 1B) for ligation to a target polynucleotide (4) where the loop adapter at each end may be identical to or different from the other. The loop adapters may have one or more complementary (double-stranded) regions (3) and at least one of the double-stranded regions may be blunt-ended (8, 9) or have an overhang (10, 11). The loop adapters also contain one or more non-complementary (single-stranded) regions (2, 5). A cleavage site (for example a modified nucleotide or bond) is shown as (X) and may be located in a double-stranded (7) or single-stranded (1) region of the adapter. X may be the same or different if present in both (1) and (7).

FIG. 2 provides an example of a loop adapter sequence (SEQ ID NO:3) containing a modified nucleotide, which is shown here as deoxyuracil (1), a region of the sequence that forms a double-stranded DNA (3), a forward primer site (12) to which primer 1 (SEQ ID NO:1) hybridizes, and a sequence (13) in the adapter whose reverse complement (15) (SEQ ID NO:4) hybridizes to a reverse primer 2 (SEQ ID NO:2).

FIG. 3 shows an outline of a method for end-repair, adapter-ligation and PCR amplification of a library of fragments of unknown sequence.

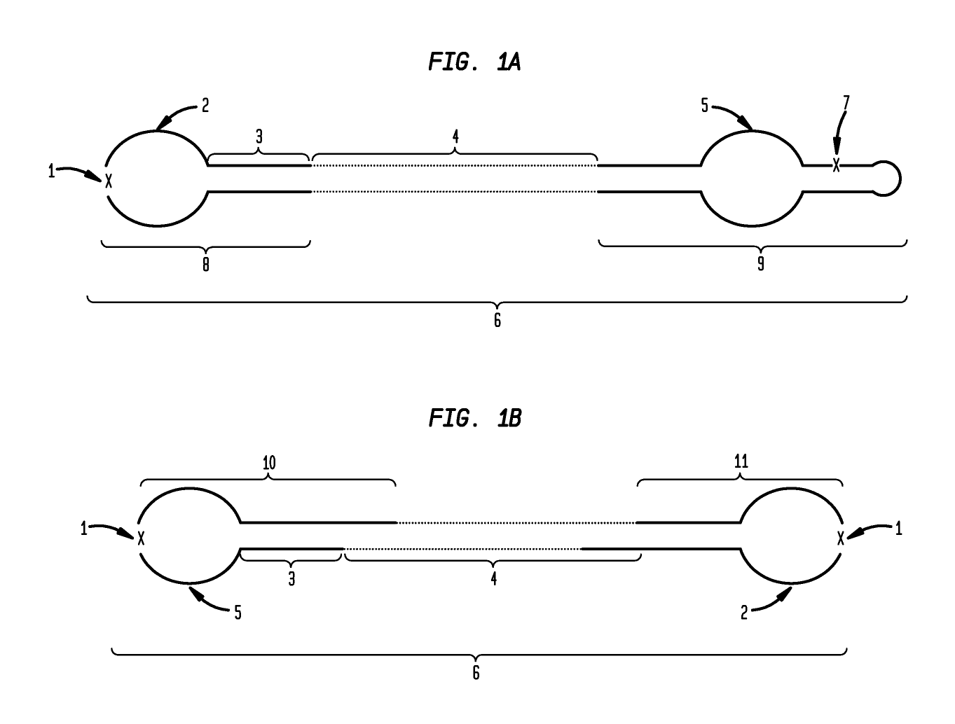

Double-stranded oligonucleotides when fragmented may have single-stranded overhangs. These fragments are end-repaired (a) to create a blunt-ended molecule (see for example, Example 2). However, a single nucleotide overhang may be added as described in Example 3. Loop adapters, e.g. (8) or (9) in FIG. 1A and (10) or (11) in FIG. 1B, are ligated onto both ends of the end-repaired fragment (b). The unligated fragments and excess loop adapters can be removed by treating with one or more DNA exonucleases. Optionally, enzymes in the reaction mixture may be heat-inactivated. The loop adapters are cleaved at a modified nucleotide or bond, or at a site which depends on the presence of a modified nucleotide or bond using an endonuclease or other cleaving enzymatic or chemical agent (c). Primers anneal to the adapters (d). The library is amplified by primer-dependent amplification (e).

FIGS. 4A-4J provide examples of loop adapters.

FIGS. 4A-4C shows blunt-ended adapters containing a single modified nucleotide (X) within a single-stranded region where FIG. 4A has no additional modification at the 5′ end; FIG. 4B has a a phosphorylated 5′ end; and FIG. 4C has an adenylated 5′ end.

FIG. 4D shows a blunt-ended adapter that has been 5′ phosphorylated and contains two modified nucleotides (X).

FIG. 4E shows a blunt-ended adapter that has been 5′ phosphorylated and contains at least one modified bond (grey region).

FIG. 4F shows a blunt-ended adapter that has been 5′ adenylated and contains modified nucleotides (X) and modified bonds (grey region).

FIG. 4G shows an adapter with a 5′ overhang, where the 5′ end has been phosphorylated and wherein the adapter contains a second double-stranded region containing two modified nucleotides (X) and an internal single-stranded loop.

FIG. 4H shows an adapter with a 5′ overhang, where the 5′ end has been phosphorylated and wherein the adapter contains a second double-stranded region containing modified bonds (grey region).

FIG. 4I shows a loop adapter with an adenylated 5′ end that contains a terminal loop and two interior loops containing modified nucleotides (X).

FIG. 4J shows a loop adapter with a 3′ overhang, a 5′ end which has been phosphorylated, a terminal single-stranded loop, an interior single-stranded loop and two modified nucleotides located within a double-stranded region.

FIGS. 5A-5E show examples of base-pairing and non-base-pairing regions in loop adapters.

FIGS. 5A-5B show a 5-nucleotide loop region within a synthetic oligonucleotide (SEQ ID NO:5 and SEQ ID NO:6.).

FIG. 5C shows a synthetic oligonucleotide folded into a loop adapter having two loop regions (SEQ ID NO:7 and SEQ ID NO:8).

FIG. 5D shows a mismatched region within a single synthetic oligonucleotide folded into a loop adapter having two loop regions (SEQ ID NO:9 and SEQ ID NO:10).

FIG. 5E shows many short mismatched regions located between many short regions of complementarity within a synthetic oligonucleotide folded into a loop adapter having two loop regions (SEQ ID NO:11 and SEQ ID NO:12).

FIG. 6 shows a 2% agarose gel in which the bands indicated by the arrow correspond to amplification products produced using a variety of adapters as described below.

Lane 1—ladder (New England Biolabs (NEB), Ipswich, Mass., #N3233)

Lanes 2 and 3 show use of Y-adapters.

Lanes 4 and 5 show use of universal loop adapters containing a modified base (uracil) not treated with the cleaving agent USER™ (NEB, Ipswich, Mass.) so that no amplification product was observed.

Lanes 6 and 7 show universal loop adapters treated with USER™, which resulted in amplification products.

FIGS. 7A and 7B show the results of end-repair and ligation of a mixture of DNA fragments of variable size as described in Example 2 using a 2100 Bioanalyzer (Agilent Technologies, Lexington, Mass.).

FIG. 7A shows the results obtained when DNA fragments were end-repaired using T4 DNA polymerase in the absence of VENT® polymerase (NEB, Ipswich, Mass.), followed by ligation with T4 DNA ligase. No ligation is detected (Lane 2). Lane 1 is a size marker.

FIG. 7B shows the results obtained when DNA fragments were end-repaired using T4 DNA polymerase in the presence of VENT® polymerase, followed by ligation with T4 DNA ligase. Efficient ligation is observed (Lanes 2 and 3). Lane 1 is a size marker.

### DETAILED DESCRIPTION OF THE EMBODIMENTS

A loop adapter is provided that is convenient and easy to use for amplifying polynucleotides (double-stranded nucleic acid molecules) in a mixture such as in a library. In one embodiment, the loop adapter does not need to be removed from the reaction mixture even if multiple, enzymatic reaction steps are involved in amplifying a polynucleotide. This makes it possible to perform amplification of a library of polynucleotides in a single vessel. Moreover, it is possible to repair DNA fragments in a library or other preparation prior to amplification as described in U.S. Pat. No. 7,700,283 and U.S. 2006-0177867 and then ligate an adapter to the repaired DNA fragments and amplify the product all in a single tube if desired.

In an embodiment of the invention, a loop adapter is preferably a single-stranded oligonucleotide having a size of approximately at least 20 nucleotides, preferably at least 25 nucleotides. The loop adapter may be ligated to a polynucleotide, which may be a double-stranded DNA, double-stranded RNA or a double-stranded DNA/RNA chimera and may contain non-nucleotide components.

Loop adapters as described herein can be utilized under any condition in which sequencing or amplification or both is desirable. For example, loop adapters described herein can be used to prepare polynucleotide libraries for sequencing reactions.

Loop adapters may be used in amplification reactions in the presence of detergents such as anionic, cationic, zwitterionic detergents or non-detergent surfactants as well as mixtures including lipids, for example, phospholipids such as 3-[(3-cholamidopropyl)dimethylammonio]-1-propanesulfonate (CHAPS). Loop adapters may be ligated to target polynucleotides such as fragments of DNA in a library.

The loop adapter contains a modified component such as, for example, a modified nucleotide or a modified bond (see FIGS. 1A and 1B). In one embodiment, the modified nucleotide or bond differs in at least one respect from deoxycytosine (dC), deoxyadenine (dA), deoxyguanine (dG) or deoxythymine (dT). Where the adapter is DNA, examples of modified nucleotides include ribonucleotides or derivatives thereof (for example: uracil (U), adenine (A), guanine (G) and cytosine(C)), and deoxyribonucleotides or derivatives thereof such as deoxyuracil (dU) and 8-oxo-guanine. Where the adapter is RNA, the modified nucleotide may be a dU, a modified ribonucleotide or deoxyribonucleotide. Examples of modified ribonucleotides and deoxyribonucleotides include abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. Examples of modified bonds include any bond linking two nucleotides or modified nucleotides that is not a phosphodiester bond. An example of a modified bond is a phosphorothiolate linkage.

Embodiments of the loop adapter can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, ribonucleases and silver nitrate. For example, cleavage at dU may be achieved using uracil DNA glycosylase and endonuclease VIII (USER™, NEB, Ipswich, Mass.) (U.S. Pat. No. 7,435,572). Where the modified nucleotide is a ribonucleotide, the adapter can be cleaved with an endoribonuclease; and where the modified component is a phosphorothiolate linkage, the adapter can be cleaved by treatment with silver nitrate (Cosstick, et al., Nucleic Acids Research 18(4):829-35(1990)).

Various structures for the loop adapter are provided in FIGS. 1A-1B, 4A-4J and 5A-5E. Generally, the loop adapter contains at least one or more self-complementary regions capable of forming a double-stranded structure (for example, see FIGS. 4E-4H). The complementary sequences may be of a length of at least 10 nucleotides at or near the 3′ and 5′ ends of the molecule. The number of nucleotides preferably available for annealing to form a double-stranded region is determined by factors that include one or more of the following: base composition of the region, the temperature at which the double-stranded structure is desired, and the salt conditions of the buffer containing the molecule (see for example, Davidson and Szybalski, Chapter 3 of “Physical and Chemical Characteristics of Lambda DNA” in Bacteriophage lambda ed., A. D. Hershey, pub. Cold Spring Harbor, Cold Spring Harbor, N.Y. (1971)).

In an embodiment of the invention, the oligonucleotide contains at least one double-stranded region or stem and at least one single-stranded loop of at least 5 nucleotides that forms a hairpin structure. The oligonucleotide preferably also contains primer sites located between the cleavage site and the proximate end of the oligonucleotide in either a single-stranded and/or a double-stranded region or both. In one embodiment, a specific cleavage site is positioned in a single-stranded loop. The primer sites may be located in the single- and/or double-stranded regions of the oligonucleotide. Various embodiments of oligonucleotide structures are shown in FIGS. 4A-4J and 5A-5E.

The terminal double-stranded structure of a loop adapter may have a blunt-ended terminus or an overhang at either the 5′ or 3′ end. Where the terminal double-stranded region has an overhang, it may be an overhang of a single base such as generated by the terminal transferase activity of Taq DNA polymerase, or more than one base, for example, sequences complementary to the cohesive ends generated by many restriction endonucleases, including, for example EcoRI, EcoRII, BamHI, HindIII, TaqI, NotI (see Roberts, R. J., et al., Nucl. Acids Res. 38: D234-D236 (2010)).

Ligation of loop adapters to target polynucleotides such as fragments of DNA in a library which have a single base overhang may be enhanced by the use of a small molecule enhancer.

Ligation may alternatively be enhanced by polishing staggered ends of a duplex polynucleotide using a mixture of polymerases where one of the polymerases is a thermostable polymerase with 3′-5′ exonuclease activity. The mixture can include, for example, T4 DNA polymerase and an archeael polymerase (see for example FIG. 7 and Example 2). A mixture of polymerases for polishing DNA ends as described herein can be used to prepare any type or number of duplex polynucleotides for ligation for example to loop adapters.

The 5′ end of the universal loop adapter may be modified to aid ligation of the adapter to a polynucleotide of interest. Modifications to the 5′ end of the adapter ligation include phosphorylation and adenylation. Modifications may be achieved by any means known in the art including methods comprising the use of T4 polynucleotide kinase for phosphorylation and T4 DNA ligase for adenylation. Modifications such as the incorporation of phosphothioate linkages may also be added to the 5′ and/or 3′ end of the adapter to resist exonuclease degradation.

The loop adapter contains at least two regions wherein the oligonucleotide does not form Watson-Crick base pairs even in conditions where other parts of the oligonucleotide can form a double-stranded structure. The resultant single-stranded regions may form a terminal loop of 5 or more nucleotides, an internal loop, or a region of mismatched bases, giving rise to one or more terminal or interior, symmetric or asymmetric loops forming a characteristic hairpin structure. These may arise because of: a mismatched region of a sequence of nucleotides (e.g. 5, 10, 20, 30, 50 nucleotides or more) followed by a region of stable base pairing (see FIG. 5D); and/or a mismatched region consisting of serial repeats of non-complementary nucleotides and complementary nucleotides wherein the non-complementary sequences can be one or many nucleotides in length and the complementary sequences may for example be 1-5 nucleotides long (see FIG. 5E).

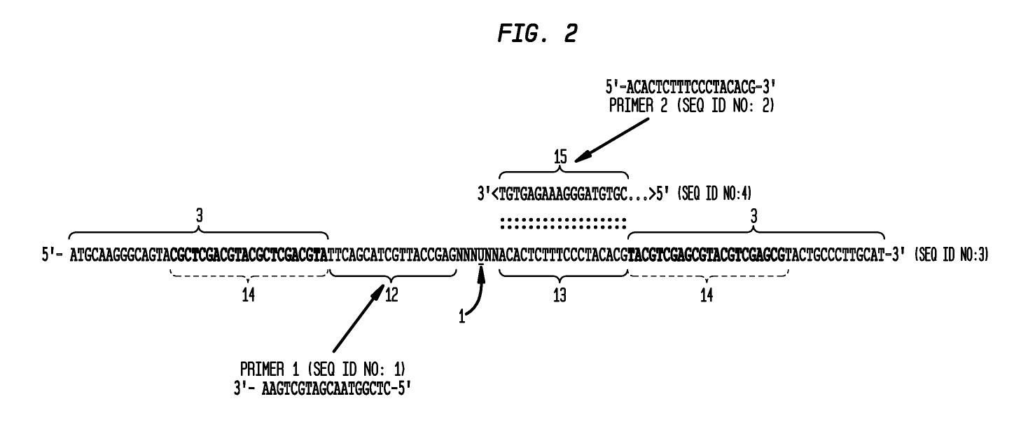

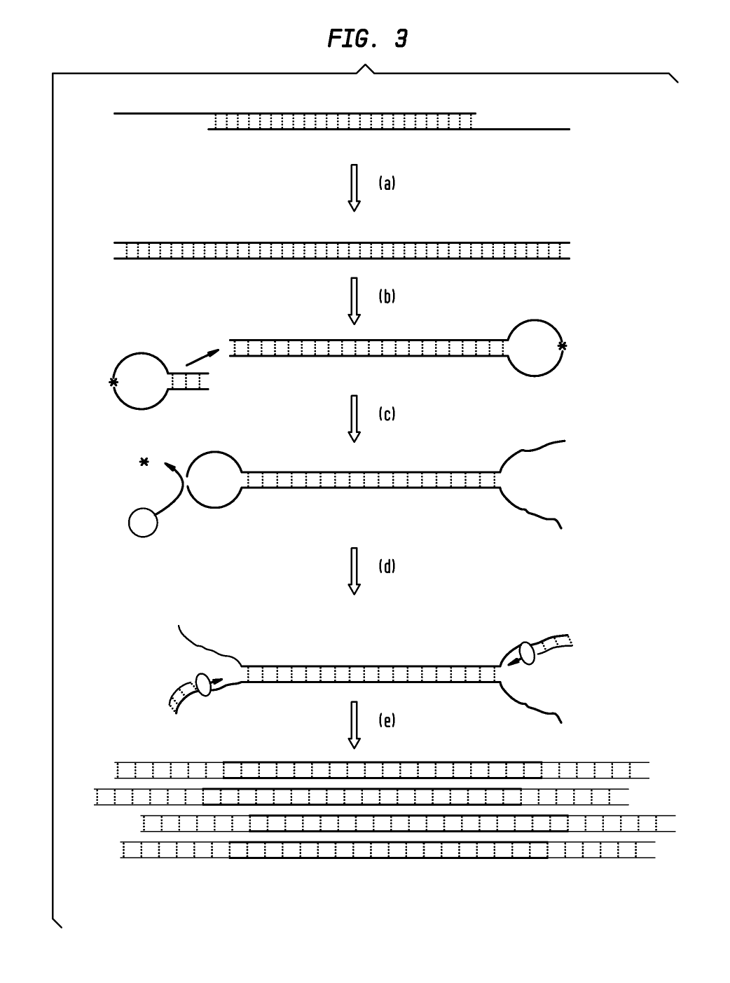

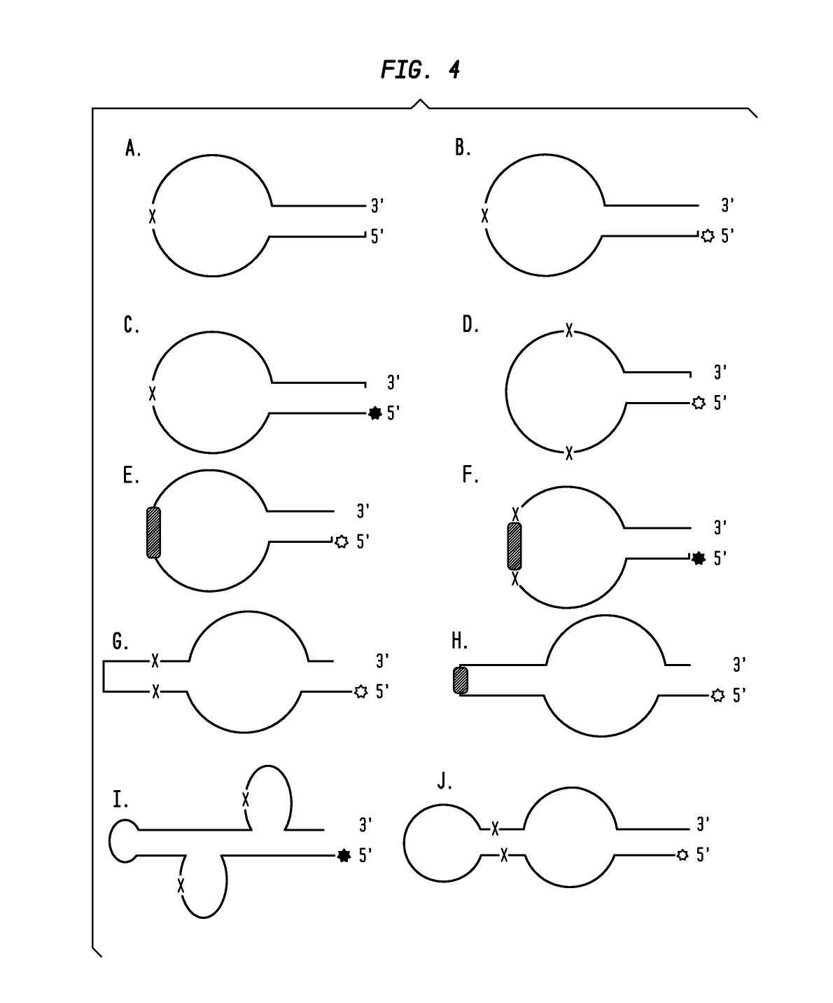

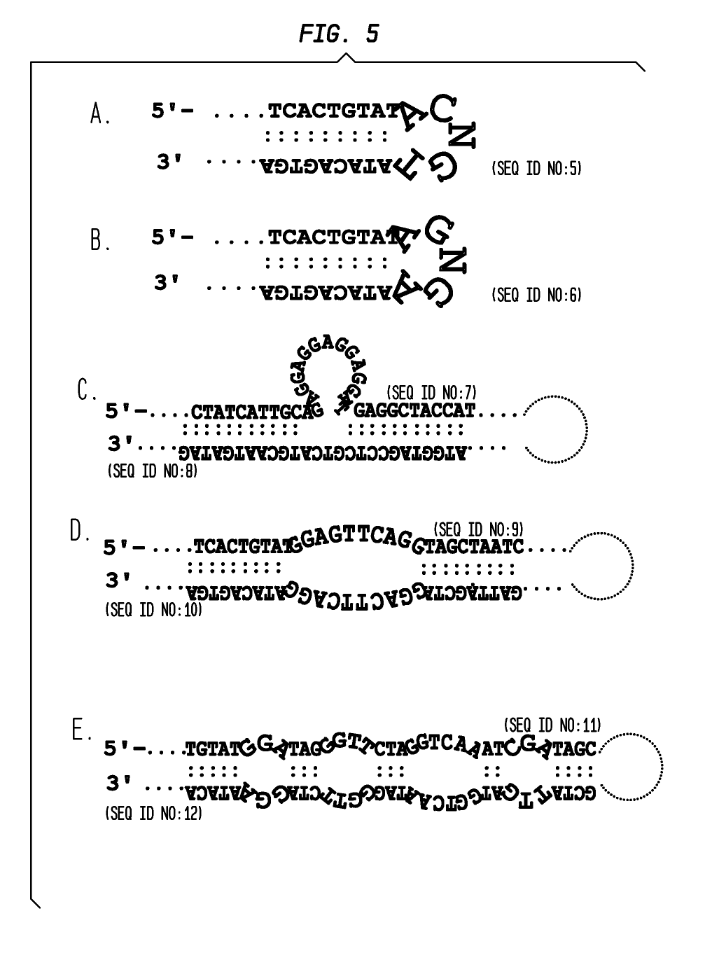

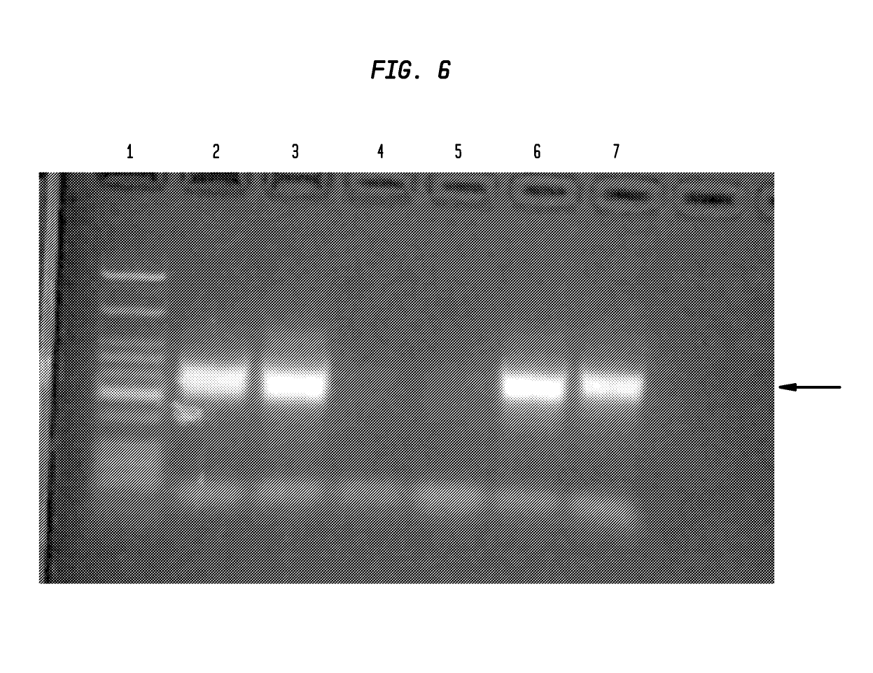

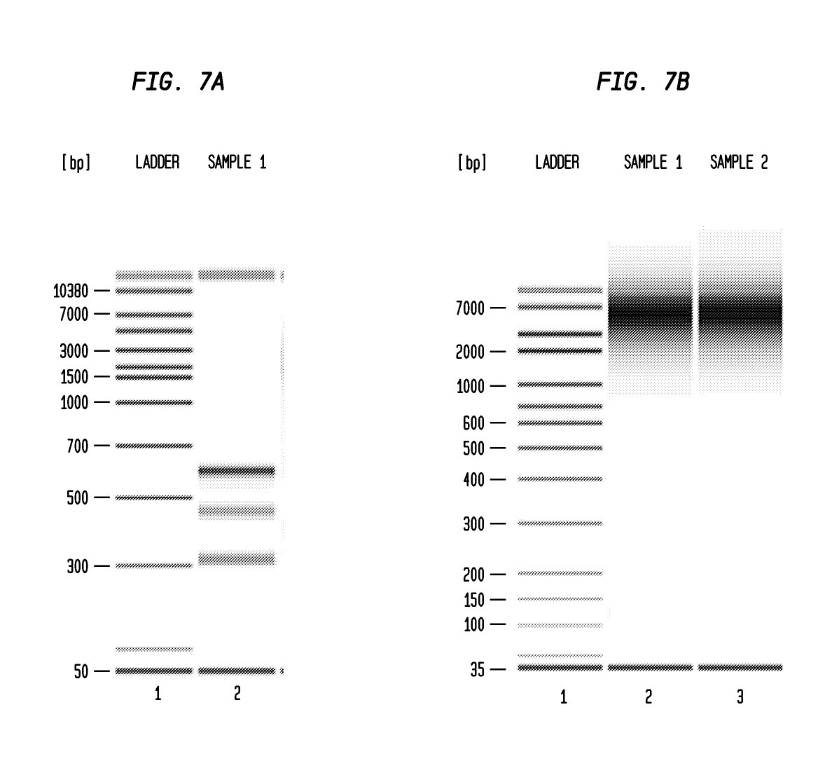

The loop adapter may contain one or more primer-associated sequences (FIGS. 2 (12) and (13)) within the adapter. The forward primer site (12) hybridizes to one or more short oligonucleotides - forward primer 5′-CTCGGTAACGATGCTGAA -3′ (SEQ ID NO:1). The reverse primer site (FIG. 2, (13)) has a reverse complement (FIG. 2, (15)) that hybridizes to a reverse primer 5′-ACACTCTTTCCCTACACG -3′ (SEQ ID NO:2). The forward and reverse primer sequences may be at least about 10 nucleotides in length and located within the single-stranded region and/or the double-stranded region of the adapter.

In an embodiment of the invention, each loop adapter contains a forward primer site and a reverse primer site where the forward primer site is located upstream and may be proximate to the reverse primer site. The forward primer may be upstream adjacent to the reverse primer site or at a distance from the reverse primer site but in both cases separated by a cleavage site. At least one modified component is preferably located between the forward primer site and the reverse primer site (for example, see FIGS. 2A-B). Alternatively or additionally, a modified component may be located within the forward primer site or the reverse primer site.

A primer may include a 5′ modification, such as an inverted base (e.g. 5′-5′ linkage); one or more phosphothioate bonds to prevent 5′-3′ exonuclease-degradation or unwanted ligation products; a fluorescent entity such as fluorescein to aid in quantification of amplification product; or a moiety, such as biotin to aid in separation of amplification product from solution.

Loop adapters may additionally include sequence identifiers such as barcodes. These may be located in the loop region or in the double-stranded region of the loop adapter. The identifier is preferably located between the terminal region of the adapter and one or more modified components.

Barcodes are preferably a sequence which is rarely found in nature (for example, see Hampikian, G. & Andersen, T. (2007) “Absent sequences: nullomers and primes” in Pacific Symposium on Biocomputing, 355-366).

Barcode sequences may be used to identify and isolate selected polynucleotides as well as to streamline downstream data analysis. A barcode can be assigned to identify specific samples, experiments or lots. Barcode sequences may be at least 2 nucleotides in length and generally no more than about 15 nucleotides in length. This provides resolution for 2⁴-15⁴ different libraries in a single mixture.

Barcodes can be used, for example, to isolate adapter-ligated polynucleotides using, for example, oligonucleotide probes. The oligonucleotide probes may be free in solution or immobilized on a matrix such that hybridization of the probe to the adapter results in a detectable signal. One or more oligonucleotide probes may be attached to a solid surface, for example, a bead, tube, or microarray.

Barcodes can be used in downstream data analysis. For example, where multiple samples comprising DNA sequences from different species are processed simultaneously, samples containing species-specific unique identifying sequences can be extracted from the raw data based on the presence of the identifier and compared to the reference genome corresponding to the species indicated in the identifying sequence. The unique identifying sequences can also be used within a quality assurance protocol, including use as a means for tracking samples through multiple reactions, personnel or processing locations.

The ligation of loop adapters to polynucleotide targets may be used in the preparation of polynucleotide libraries. A polynucleotide library may contain non-identical polynucleotides wherein at least one member of the library must contain at least one polynucleotide consisting of a sequence which differs by at least one nucleotide from one or more polynucleotides in the library.

Advantages of using loop adapters during preparation of the library include their resistance to denaturation under conditions used to denature enzymes in a reaction mix (for example, in subsequent ligation steps for mate-pair library construction); and/or enzymatic degradation of non-ligated adapters.

Under denaturing conditions including heat, chemical or enzymatic denaturing conditions, the loop adapter ligated to a target double-stranded oligonucleotide forms a substantially single-stranded circular polynucleotide. The presence of the loop adapters facilitates the renaturation of the double-stranded target oligonucleotide once the denaturing condition is reversed. This may permit many steps of polynucleotide library preparation to be optionally performed in a single reaction vessel.

Library preparation often requires intermediate purification steps between enzymatic reactions such as adapter ligation and amplification in order to reduce unwanted side reactions contaminating subsequent steps. An alternative to purification is heat-inactivation. The temperature and time requirements for reducing or eliminating the activity of enzymes are known in the art and are generally listed on data cards provided by commercial suppliers of enzymes. For example, an enzyme may be substantially inactivated by treatment at 50° C., 55° C., 60° C., 65° C., 70° C., 75° C. or 80° C. or more for 5, 10, 15, 20 or more minutes.

The circular structure of an adapter-ligated double-stranded polynucleotide target also renders the construct resistant to exonuclease-degradation. Enzymatic degradation of non-ligated adapters without damaging the ligated adapters and subsequent denaturation of the degradative enzymes in a single step provides significant advantages in efficiency and cost.

All references cited herein, including U.S. provisional applications Ser. No. 61/365,687 filed Jul. 19, 2010 and Ser. No. 61/381,767 filed Sep. 10, 2010, are incorporated by reference.

### EXAMPLES

Unless otherwise noted all reagents were obtained from NEB, Ipswich, Mass.

### Example 1

### Preparation of Loop Adapters

Oligonucleotide synthesis was carried out by addition of nucleotide residues to the 5′-terminus of the growing chain until the desired sequence was assembled using the phosphoramidite method described by Beaucage and Iyer Tetrahedron 48: 2223 (1992).

### Example 2

### Ligation of Loop Adapters to Target DNA

Ligation of loop adapters to target DNA was performed as follows:

Random DNA molecules of varying sizes and unknown sequences were first end-repaired. 220 ng of DNA fragments (10 μl) were mixed with 3.5 μl NEBNext® End Repair Buffer (E6050), 2 μl NEBNext End Repair Enzyme Mix (E6050), 0.5 μl, VENT® DNA polymerase (MO254) 3′-5′ exo+; and 19 μl water; in a total volume of 35 μl (NEB, Ipswich, Mass.).

The mixture was combined in a reaction vessel, vortexed to mix, incubated for 20 minutes at 25° C. then 20 mins at 70° C. and then returned to 25° C.

The end-repaired DNA fragments were then ligated to each other as follows:

35 μl random DNA fragments of varying sizes and unknown sequences was mixed with 10 μl NEBNext® 5×Quick Ligation Reaction Buffer (E6056); 5 μl Quick T4 DNA Ligase (NEB, Ipswich, Mass., E6056); 50 μl total volume.

The mixture was vortexed and incubated an additional 30 min at 25° C. The samples were cleaned up on Qiagen Minelute® column (Valencia, Calif.), eluted in 10 μl NEB Buffer and run twice on an Agilent Technologies High Sensitivity DNA Chip (Lexington, Mass.).

Sample 1 in FIG. 7A shows a mixture of unligated fragments in the presence of T4 DNA polymerase only whereas Sample 1 and Sample 2 in FIG. 7B to which a second thermostable polymerase was added (VENT® polymerase) were fully ligated to adapters as evidenced by a high molecular weight band.

### Example 3

### Use of Loop Adapters in Library Preparation for Sequencing

A library was prepared from starting material: 0.5 μg DNA which had been fragmented to 100-800 by by NEBNext® double-stranded DNA Fragmentase™ (NEB, Ipswich, Mass.) in 16 μl of TE.

2.5 μl NEBuffer 2 (10×), 2.5 μl ATP, 1.0 μl dNTP Mix, 1.0 μl T4 DNA Polymerase, 1.0 μl T4 polynucleotide kinase, 1.0 μl Taq DNA polymerase, 1.0 μl Quick T4 DNA Ligase, 1.0 μl universal loop adapter (SEQ ID NO:3) were mixed together in a reaction mixture. The reaction was incubated at 20° C. for 30 minutes, then incubated at 72° C. for 20 minutes. The reaction mixture was then added to Agencourt® Ampure® magnetic beads (Beckman Coulter, Brea, Calif.), vortexed and incubated at room temperature for 5 minutes. In the presence of a magnet, the beads were then separated from the supernatent which was discarded.

The beads were washed twice in Tris-EDTA (TE) followed by NEBNext® Sizing Buffer and ethanol and then re-suspended. Using a magnet, the beads were separated from the supernatent and the supernatent was collected. 50 μL of the supernatant (containing the adapter-ligated DNA library) was treated with 1 μl of exonuclease mix. The reaction was held at 20° C. for 10 minutes, then incubated at 72° C. for 20 minutes. The adapter-ligated polynucleotide library was then treated with 5 μl of USER™ enzyme mix.

The library may then be sequenced using a NextGen sequencing platform such as 454 (Roche, Branford, Conn.) or Illumina (San Diego, Calif.) GAIIx or HiSEQ ESFLX.

### Example 4

### Alternate Use of Loop Adapters for Library Preparation for Sequencing

A library was prepared from starting material: 1-5 pg DNA was fragmented to 100-800 by by NEBNext® double-stranded DNA Fragmentase™ (New England Biolabs) and was mixed with 20 μl NEBNext® End Repair Reaction Buffer (10×), 10 μl NEBNext End Repair Enzyme Mix, sterile H₂O in amount as necessary to make a final volume of 200 μl. The reaction mixture was placed in a thermal cycler for 15 minutes at 20° C. and the DNA was column-purifed.

The purified DNA was added to 40 μl NEBNext Quick Ligation Reaction Buffer (5×), variable loop adapters containing deoxyuracil, 10 μL T4 DNA Ligase and sterile H₂O in amount as necessary to make a final volume of 200 μl. The reaction mixture was placed in a thermal cycler for 15 minutes at 20° C. The adapter-ligated polynucleotide library was treated with 1 μl of exonuclease mix. The reaction was incubated at 20° C. for 10 minutes, then incubated at 72° C. for 20 minutes. The adapter-ligated polynucleotide library was then treated with 5 μl of USER™, held at 20° C. for 10 minutes, then incubated at 72° C. for 20 minutes.

The exonuclease-enriched adapter-ligated DNA library was added to: 10 μl forward primer (50 μM stock), 10 μl reverse primer (50 μM stock), 250 μl LongAmp Taq 2×Master Mix, and sterile H₂O in amount as necessary to make a final volume of 500 μl.

125 μL aliquots of the above mixture was then PCR amplified and the amplification product column purified for sequencing on a NextGen platform SOLiD™ (Applied Biosystems, no Life Technologies, Carlsbad, Calif.) or cloning or other type of analysis.

### Example 5

### Alternate Use of Loop Adapters for Library Preparation for Sequencing

A library was prepared as follows: 1-5 μg DNA was fragmented to 100-800 by by NEBNext® Fragmentase™ in less than 85 μl of TE.

The library of fragments were end-repaired by adding to 10 μl NEBNext® End Repair Reaction Buffer (10×), 5 μl NEBNext End Repair Enzyme Mix, and sterile water to a final volume of 100 μl. The reaction mixture was incubated in a thermal cycler for 30 minutes at 20° C. The end-repaired DNA sample was column-purified and eluted in 37 μl of sterile H₂O or elution buffer.

The 37 μl end-repaired, blunt DNA was added to 5 μl NEBNext dA-Tailing Reaction Buffer (10×), 3 μl Klenow fragment (3′-5′ exo⁻) and 5 μl sterile H₂O. This reaction was incubated in a thermal cycler for 30 minutes at 37° C. The DNA sample was column-purified and eluted in 25 μl of sterile H₂O or elution buffer.

The 25 μl end-repaired, dA-tailed DNA was added to 10 μl Quick Ligation Reaction Buffer (5×), 10 μM loop adapters, 5 μl T4 DNA Ligase and was incubated in a thermal cycler for 15 minutes at 20° C. The adapter-ligated polynucleotide library was treated with 1 μl of exonuclease mix. The adapter-ligated polynucleotide library was then treated with a cleaving agent, held at 20° C. for 10 minutes, then incubated at 72° C. for 20 minutes. To the exonuclease-enriched adapter-ligated DNA library, 1 μl forward primer (10 μM stock), 1 μl reverse primer (10 μM stock), 1 μl dNTP Mix and 1 U Phusion® (Thermo Fisher Scientific, Waltham, Mass.) High-Fidelity Polymerase was added and subjected to PCR. The DNA was then column-purified and used for sequencing using a Next Generation platform such as Illumina EAIIx or HiSeq or for other desired analyses or uses.

What is claimed is:

1. A composition, comprising: a synthetic oligonucleotide characterized by a double-stranded region, a single-stranded region, a forward primer site, a reverse primer site and one or more cleavage sites therebetween.

2. The composition according to claim 1, wherein the synthetic oligonucleotide has a sequence that permits the oligonucleotide to fold into a structure having at least one single-stranded loop and one double-stranded region where the double-stranded region has a 3′ end and a 5′ end.

3. The composition according to claim 2, wherein the 3′ end and the 5′ end form a blunt end or a staggered end.

4. The composition according to claim 1, wherein the one or more cleavage sites comprise a modified nucleotide or a sequence containing a modified or unmodified nucleotide that is specifically recognized by a cleavage agent, a chemical group, a chemical linker or a spacer.

5. The composition according to claim 1, wherein the forward primer site is positioned between the 3′ end and a proximate cleavage site and wherein the reverse primer site is positioned between the 5′ end and the same or different proximate cleavage site.

6. The composition according to claim 5, wherein the 5′ end is phosphorylated or adenylated.

7. The composition according to claim 1, wherein the oligonucleotide further comprises a barcode sequence which is adjacent to a primer site.

8. The composition according to claim 7, wherein the barcode is 2-15 nucleotides in length.

9. The composition according to claim 1, wherein the oligonucleotide is ligated to a polynucleotide having an unknown sequence.

10. The composition according to claim 7, wherein the barcode is adjacent to the polynucleotide.

11. The composition according to claim 1, wherein the oligonucleotide is capable of use as an adapter for amplification and/or sequencing reactions.

12. The composition according to claim 1, wherein the oligonucleotide is resistant to exonuclease-degradation.

13. The composition according to claim 1, wherein the oligonucleotide remains non-denatured during an enzyme-denaturing temperature.

14. A method for preparing polynucleotides for ligation, comprising: blunt-ending a polynucleotide using an enzyme mixture comprising T4 DNA polymerase and a thermostable polymerase having 3′-5′ exonuclease activity such that the blunt-ended polynucleotide is capable of being ligated to an oligonucleotide according to claim 1.

15. The method according to claim 14, wherein the thermostable polymerase is an archaeal polymerase.

16. The method according to claim 14, further comprising: ligating by means of a ligase, the oligonucleotide to each end of the blunt-ended polynucleotide.

17. The method according to claim 16, further comprising cleaving the ligated oligonucleotide with a cleaving agent.

18. A method of preparing an amplification-ready polynucleotide in a reaction vessel, comprising:
ligating by means of a ligase, a composition according to claim 1 to each end of the polynucleotide;
treating the preparation of ligation products with a cleaving agent, wherein the cleaving agent cleaves the oligonucleotide at or near the one or more modified components in the oligonucleotide; and
amplifying the polynucleotide.

19. The method according to claim 18, wherein a nuclease is added to the reaction vessel to degrade unligated polynucleotides and/or unligated oligonucleotides.

20. The method according to claims 18, wherein the reaction vessel is heated to an enzyme-denaturing temperature suitable for denaturing the degrading enzyme and/or ligase.

21. The method according to claim 18, wherein the method is performed in a single reaction vessel.


SEQUENCE LISTING (12 sequences)

SEQ ID NO: 1 - DNA, 18 bp, artificial
  primer
ctcggtaacgatgctgaa

SEQ ID NO: 2 - DNA, 18 bp, artificial
  primer
acactctttccctacacg

SEQ ID NO: 3 - DNA, 114 bp, artificial
  synthetic loop adapter
atgcaagggcagtacgctcgacgtacgctcgacgtattcagcatcgttaccgagnnnunnacactctttccctacacgtacgtcgagcgtacgtcgagcgtactgcccttgcat

SEQ ID NO: 4 - DNA, 18 bp, artificial
  primer
tgtgagaaagggatgtgc

SEQ ID NO: 5 - DNA, 23 bp, artificial
  synthetic loop adapter
tcactgtatacngtatacagtga

SEQ ID NO: 6 - DNA, 23 bp, artificial
  synthetic loop adapter
tcactgtatagngaatacagtga

SEQ ID NO: 7 - DNA, 37 bp, artificial
  synthetic loop adapter
ctatcattgcagaggaggaggaggatgaggctaccat

SEQ ID NO: 8 - DNA, 26 bp, artificial
  synthetic loop adapter
atggtagcctcgtcatgcaatgatag

SEQ ID NO: 9 - DNA, 28 bp, artificial
  synthetic loop adapter
tcactgtatggagttcaggtagctaatc

SEQ ID NO: 10 - DNA, 28 bp, artificial
  synthetic loop adapter
gattagctaggacttcaggatacagtga

SEQ ID NO: 11 - DNA, 33 bp, artificial
  synthetic loop adapter
tgtatggatagggttctaggtcaaatcgatagc

SEQ ID NO: 12 - DNA, 33 bp, artificial
  synthetic loop adapter
gctattgatggtcaatagggttctaggaataca